(19) United States
(12) Patent Application Publication
(10) Pub. No.: US 2009/0081174 A1
(43) Pub. Date: Mar. 26, 2009

(54) Synergistic fungicidal compositions

(76) Inventors: Lucio Filippini, San Donato Milanese-Milano (IT); Marilena Gusmeroli, Monza-Milano (IT); Silvia Mormile, Novara (IT); Carlo Garavaglia, Cuggiono-Milano (IT); Luigi Mirenna, Milano (IT)

Correspondence Address: HEDMAN & COSTIGAN P.C., 1185 AVENUE OF THE AMERICAS, NEW YORK, NY, 10036, US

(21) Appl. No.: 11/921,031

(22) PCT Filed: May 29, 2006
(86) PCT No.: PCT/EP2006/005157

§ 371 Date: Dec. 19, 2007

(30) Foreign Application Priority Data
May 31, 2005 (IT) MI2005A001019

(51) Int. Cl. A61K 35/74; A61K 31/7008

(52) U.S. Cl. 424/93.45; 514/62

(57) ABSTRACT

Fungicidal compositions are described, consisting of mixtures comprising a copper (II) salt of phosphorous acid and at least another metallic salt of phosphorous acid or consisting of mixtures comprising a copper (II) salt of phosphorous acid, alone or mixed with at least another metallic salt of phosphorous acid, and one or more fungicidal compounds, and their use for the control of phyto-pathogen fungi.

The present invention relates to synergistic fungicidal compositions.

In particular, the present invention relates to new synergistic compositions capable of controlling phytopathogens which cause considerable economic damage to agricultural crops.

More specifically, the present invention also relates to the use of compositions comprising mixtures based on a copper (II) salt of phosphorous acid, also called phosphite or phosphonate, with at least another metallic salt of phosphorous acid and the use of compositions comprising mixtures based on a copper (II) salt of phosphorous acid, also called phosphite or phosphonate, alone or mixed with at least another salt of a metal of phosphorous acid and one or more fungicidal compounds.

The use of salts of phosphorous acid as fungicides is generally known in literature, for example in American patent U.S. Pat. No. 4,075,324.

The Applicant has now surprisingly found that copper (II) salts of phosphorous acid are capable of exerting a considerable synergistic effect allowing a higher fungicidal activity to be obtained, than that envisaged on the basis of the activities of single components, when applied in composition with other fungicides, in addition to providing excellent anti-resistance properties.

As resistance phenomena can arise in nature as a result of prolonged treatment with systemic fungicides, it is always important and necessary to find synergies oriented towards anti-resistance strategies.

More specifically, the Applicant has now surprisingly found that mixtures based on copper (II) salts of phosphorous acid and one or more salts of phosphorous acid obtained from metals other than copper, have a considerable synergistic effect, allowing a higher fungicidal activity to be obtained than that envisaged on the basis of the activities of single components and are capable of effectively controlling numerous diseases which can cause damage to crops of great economic interest, such as, vines, potatoes and tobacco.

Said mixtures also have an excellent toxicological and environmental profile, as well as the advantage of having a wider range of use and allowing the dosages of use of copper and also, possibly, of phosphorous acid, to be reduced.

Furthermore, the Applicant has surprisingly found that said mixtures of copper (II) phosphite and other metallic phosphites, in addition to copper (II) phosphite alone, in turn demonstrate a significant synergy with numerous fungicidal products, having common biological targets with those of said copper (II) salt of phosphorous acid.

An object of the present invention therefore relates to a fungicidal composition consisting of mixtures comprising a copper (II) salt of phosphorous acid and at least another metallic salt of phosphorous acid or consisting of mixtures comprising a copper (II) salt of phosphorous acid, alone or mixed with at least another metallic salt of phosphorous acid, and one or more fungicidal compounds.

In particular, the metallic salt of phosphorous acid can be a salt of an alkaline, alkaline-earth metal, an ammonium salt or a salt of Fe, Mn, Zn, Ni, Al, Ti or Se.

Within the spirit of this invention, said mixtures comprising a copper (II) salt of phosphorous acid and at least another metallic salt of phosphorous acid, can be obtained either by mixing the single salts, or by co-precipitation of said salts in a reaction mixture; in both cases, in the above mixture, the copper (II) salt of phosphorous acid and the other metallic salts of phosphorous acid can be present in any proportion, salvation state, structure and crystalline lattice composition.

The salts of an alkaline, alkaline-earth metal, ammonium salt or salts of Fe, Mn, Zn, Ni, Al, Ti or Se, of phosphorous acid can be mono- or dibasic, or they can be mixtures of these in any proportion.

In the fungicidal compositions according to the present invention, said mixtures can also contain salts of alkaline metals in percentages not higher than 10% by weight, such as reaction exchange salts which however do not have a direct fungicidal activity.

As already mentioned, an extension of said synergy concept is to obtain mixtures comprising a copper (II) salt of phosphorous acid alone or in a mixture with at least another metallic salt of phosphorous acid, and with other fungicidal compounds have common biological targets with those of said copper (II) salt of phosphorous acid.

The compound having a fungicidal activity according to the present invention is preferably selected from:
(1) IR5885, a dipeptide compound corresponding to diastereoisomeric mixtures of methyl [S—(R,S)]-[3-(N-isopropoxycarbonylvalinyl)-amino]-3-(4-chlorophenyl)propanoate in any proportion, or to one of the two diastereoisomeric forms S—R or S—S taken individually;
(2) IR6141, corresponding to N-(phenylacetyl)-N-2,6-xylyl-R-methyl alaninate;
(3) Salicylic acid (SA) or its derivatives such as acetylsalicylic acid (ASA), copper salts of salicylic acid or acetylsalicylic acid;
(4) A copper (I) or copper (II) salt, such as copper oxychloride, copper hydroxide, Bordeaux mixture, copper sulfate, or a mixture of copper hydroxide and oxychloride (Airone);
(5) Benalaxyl corresponding to N-(phenylacetyl)-N-2,6-xylyl-RS-methyl alaninate;
(6) Metalaxyl corresponding to N-(2-methoxyacetyl)-N-2,6-xylyl-RS-methyl alaninate;
(7) Metalaxyl-M corresponding to N-(2-methoxyacetyl)-N-2,6-xylyl-R-methyl alaninate;
(8) Oxadixyl corresponding to 2-methoxy-N-(2-oxo-1,3-oxazolidin-3-yl)aceto-2′,6′-xylidide;
(9) Ofurace corresponding to DL-3-[N-chloroacetyl-N-(2,6-xylyl)-amino]-γ-butyrolactone;
(10) Iprovalicarb corresponding to O-(1-methyl-ethyl)-N-[2-methyl-1-[[[1-(4-methyl-phenyl)ethyl]amino]carbonyl]propyl]carbamate;
(11) Benthiavalicarb-isopropyl corresponding to O-isopropyl [(S)-1-{[(1R)-1-(6-fluoro-1,3-benzothiazol-2-yl)ethyl]-carbamoyl-2-methylpropyl}-carbamate;
(12) Cymoxanil corresponding to 1-(2-cyano-2-methoxyimino-acetyl)-3-ethylurea;
(13) Azoxystrobin corresponding to (E)-2-(2-[6-(2-cyanophenoxy)-pyrimidin-4-yloxy]phenyl-3-methyl methoxyacrylate;
(14) Metominofen corresponding to N-methyl-(E)methoxyimino-(2-phenoxyphenyl)acetamide;
(15) Pyraclostrobin corresponding to methyl N-(2-[1-(4-chlorophenyl)pyrazol-3-yloxymethyl]-phenyl)-N-methoxycarbamate;
(16) Acibenzolar-5-methyl corresponding to methyl benzo(1,2,3)thiadiazole-7-thiocarboxylate;
(17) Famoxadone corresponding to 5-methyl-5-(4-phenoxyphenyl)-3-(phenylamino)oxazolidin-2,4-dione;
(18) Fenamidone corresponding to 4-methyl-4-phenyl-1-(phenylamino)-2-methylthioimidazolidin-5-one;
(19) Cyazofamide, corresponding to 2-cyano-4-chloro-5-(4-methylphenyl)-1-(N,N dimethylaminosulfamoyl)imidazole;
(20) Fluazinam corresponding to 3-chloro-N-(3-chloro-5-trifluoromethyl-2-pyridyl)-α-α-α-trifluoro-2,6-dinitro-ρ-toluidine;
(21) Dimethomorph corresponding to (E,Z)-4-[3-(4-chlorophenyl)-3-(3,4-dimethoxyphenyl)-acryloyl]morpholine; or Flumorph (SYP-L190) corresponding to (E,Z)-4-[3-(4-fluorophenyl)-3-(3,4-dimethoxyphenyl)-acryloyl]morpholine;
(22) Flumetover corresponding to 2-(3,4-dimethoxyphenyl)-N-ethyl-α,α.α-trifluoro-N-methylp-toluamide;
(23) Chlorothalonil corresponding to 1,3-dicyano-2,4,5-tetrachlorobenzene;
(24) Mancozeb corresponding to the manganese and zinc salt of ethylenebis(dithiocarbamate) (polymer);
(25) Tolylfluanide corresponding to N-dichlorofluoromethylthio-N′,N′-dimethyl-N-p-tolylsulfamide;
(26) Folpet corresponding to N-(trichloromethylthio)phthalimide;
(27) Etridiazole corresponding to ethyl-3-trichloromethyl-1,2,4-thiadiazolyl ether;
(28) Hymexanol corresponding to 5-methylisooxazol-3-ol;
(29) Propamocarb corresponding to propyl-(3-dimethylaminopropyl)carbamate;
(30) R-3-aminobutanoic acid or RS-3-aminobutanoic acid;
(31) Zoxamide, corresponding to 3,5-dichloro-N-(3-chloro-1-ethyl-1-methyl-2-oxopropyl)-p-toluamide;
(32) 2-(4-chlorophenyl)-N-[2-(3-methoxy-4-prop-2-inyloxy-phenyl)ethyl]-2-prop-2-inyloxy-acetamide;
(33) Ethaboxam, (RS)-α-cyano-2-thienyl)-4-ethyl-2(ethylamino)-5-thiazolecarboxyamide;
(34) Saccharin

The compounds (1) are described in Italian patent application Nr. MI98A002583.

Compound (2) is described in patent application WO 98/26654 A2.

The compounds (3) are commercial products and their copper salts are described in Italian patent application Nr. MI 2001A002430.

The compounds (4) are easily available on the market.

Compound (5) is described in “The Pesticide Manual”, 1983, VIIth edition, British Crop Protection Council Ed., page 32.

Compound (6) is described in English patent GB 1,500,581.

Compound (7) is described in patent application Wo 96/01559 A1.

Compound (8) is described in English patent GB 2,058,059.

Compound (9) is described in “Phytopathological News” (1978), Vol. 9, page 142.

Compound (10) is described in patent applications EP 550,788 and EP 775,696.

Compound (11) is described in patent application EP 775,696.

Compound (12) is described in “The Pesticide Manual”, 1983, VIIth edition, British Crop Protection Council Ed., page 148.

Compound (13) is described in European patent application EP 382,375.

Compound (14), corresponding to the experimental abbreviation SSF-126, is described in U.S. Pat. No. 5,185,242.

Compound (15) is described in patent application WO 96/01258.

Compound (16) is described in U.S. Pat. No. 4,931,581.

Compound (17) is described in “Brighton Crop Protection Conference—Pests and Diseases” 1996, Congress Records.

Compound (18) is described in European patent application EP 629,616.

Compound (19), also called IKF916, is described in European patent application EP 705,823.

Compound (20) is described in European patent application EP 31,257.

The compounds (21) are described respectively in European patent application EP 219,756 and in “Brighton Crop Protection Conference—Pests and Diseases” 2000, Congress Records.

Compound (22) is described in European patent applications EP 360,701 and EP 611,232.

Compound (23) is described in “The Pesticide Manual”, 1983, VIIth edition, British Crop Protection Council Ed., page 120.

Compound (24) is described in “The Pesticide Manual”, 1983, VIIth edition, British Crop Protection Council Ed., page 339.

Compound (25) is described in “The Pesticide Manual”, 1983, VIIth edition, British Crop Protection Council Ed., page 537.

Compound (26) is described in “The Pesticide Manual”, 1983, VIIth edition, British Crop Protection Council Ed., page 599.

Compound (27) is described in “The Pesticide Manual”, 1983, VIIth edition, British Crop Protection Council Ed., page 252.

Compound (28) is described in “The Pesticide Manual”, 1983, VIIth edition, British Crop Protection Council Ed., page 314.

Compound (29) is described in “The Pesticide Manual”, 1983, VIIth edition, British Crop Protection Council Ed., page 471.

Compound (30) is described in European patent application EP 753,258.

Compound (31) is described in “Brighton Crop Protection Conference—Pests and Diseases” 1998, Congress Records.

Compound (32) is described in patent application WO 01/87822.

Compound (33) is described in “The Pesticide Manual”, 2003, XIIIth edition, British Crop Protection Council Ed.

Compound (34) is a commercial product.

Preferred fungicidal compositions according to the present invention can be selected from
Mixture 1: CuHPO₃+CaHPO₃ (50:50)
Mixture 2: CuHPO₃+ZnHPO₃ (50:50)
Mixture 3: CuHPO₃+MnHPO₃ (50:50)
Mixture 4: CuHPO₃+MnHPO₃ (66:33)
Mixture 100: Mixture 1+IR5885 (750⁽¹⁾:15⁽²⁾)
Mixture 103: Mixture 3+IR5885 (750⁽¹⁾:15⁽²⁾)
Mixture 105: Mixture 2+IR5885 (750⁽¹⁾:15⁽²⁾)
Mixture 124: CuHPO₃+IR5885 (750⁽¹⁾:15⁽²⁾)
Mixture 125: CuHPO₃+IR6141 (750⁽¹⁾:15⁽²⁾)
Mixture 126: Mixture 1+IR6141 (750⁽¹⁾:15⁽²⁾)
Mixture 129: Mixture 3+IR6141 (750⁽¹⁾:15⁽²⁾)
Mixture 131: Mixture 2+IR6141 (750⁽¹⁾:15⁽²⁾)
Mixture 143: CuHPO₃+Cu(SA) (750⁽¹⁾:64⁽³⁾)
Mixture 144: Mixture 1+Cu(SA) (750⁽¹⁾:64⁽³⁾)
Mixture 145: Mixture 3+Cu(SA) (750⁽¹⁾:64⁽³⁾)
Mixture 146: Mixture 2+Cu(SA) (750⁽¹⁾:64⁽³⁾)
Mixture 152: CuHPO₃+Cu(ASA)₂ (750⁽¹⁾:64⁽³⁾)
Mixture 153: Mixture 1+Cu(ASA)₂ (750⁽¹⁾:64⁽³⁾)
Mixture 154: Mixture 3+Cu(ASA)₂ (750⁽¹⁾:64⁽³⁾)
Mixture 155: Mixture 2+Cu(ASA)₂ (750⁽¹⁾:64⁽³⁾)
Mixture 361: CuHPO₃+Saccharin (750⁽¹⁾:125⁽²⁾)
Mixture 362: Mixture 1+Saccharin (750⁽¹⁾:125⁽²⁾)
Mixture 363: Mixture 2+Saccharin (750⁽¹⁾:125⁽²⁾)
Mixture 364: Mixture 3+Saccharin (750⁽¹⁾:125⁽²⁾)
   (1): dosage expressed in ppm of equivalent phosphorous acid.
   (2): dosage expressed in ppm of active ingredient
   (3): dosage expressed in ppm of equivalent metallic copper.

The fungicidal compositions defined as mixture 1, mixture 2, mixture 3 and mixture 4 are particularly preferred.

When they are not commercial products, the single metallic salts of phosphorous acid can be easily obtained in aqueous solution by reaction of phosphorous acid with an inorganic base such as the hydroxide or carbonate of the desired metal, or by reaction of phosphorous acid with an inorganic base, such as sodium or potassium hydroxide and the subsequent reaction with an inorganic salt, for example, chloride, bromide or sulfate of the desired metal, as described in literature in: “GMelins Handbuch Der Anorganischen Chemie” Ed. Verlag Chemie—GMBH—Weinheim.

The fungicidal compositions according to the present invention can be prepared as follows: the mixtures comprising a copper (II) salt of phosphorous acid and at least another metallic salt of phosphorous acid are obtained by mixing the single salts or by co-precipitation of said salts in the reaction mixture.

In this second case, the co-precipitation of the salts is effected starting from phosphites, for example sodium, potassium phosphite, by the shifting of the metallic cation M¹ with a mixture, in aqueous solution, of a copper (II) salt and the other desired metallic salt, according to scheme A:

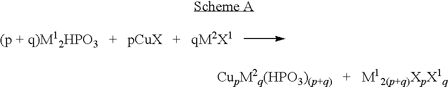

wherein M¹ represents a sodium or potassium atom, X represents a halogen, such as chlorine, bromine, iodine or a sulfate group, X¹ has the same meaning as X, M² represents an alkaline earth metal, an ammonium group or a Fe, Mn, Zn, Ni, Al, Ti, Se atom; p is a number greater than 0 and lower than 100 and q is equal to 100-p.

The co-precipitation of a copper (II) salt of phosphorous acid and a salt of an alkaline metal of phosphorous acid is effected starting from the above alkaline salt of phosphorous acid by partial shifting of the metallic cation with a suitable copper (II) salt according to scheme B:

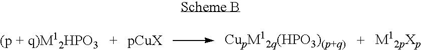

wherein M¹, X, p and q have the meanings indicated above.

Analogously mixtures comprising, in addition to copper, two or more metals selected from those listed above, can be prepared.

The fungicidal compositions according to the present invention comprising:

a) a copper (II) salt of phosphorous mixed with at least one salt of an alkaline, alkaline earth metal, an ammonium salt or a salt of Fe, Mn, Zn, Ni, Al, Ti, Se of phosphorous acid;
b) a copper (II) salt of phosphorous acid alone or in a mixture with at least one salt of an alkaline, alkaline earth metal, an ammonium salt, or a salt of Fe, Mn, Zn, Ni, Al, Ti, Se of phosphorous acid, and at least one of the fungicidal compounds (1)-(34);
all have a high fungicidal activity with respect to numerous fungal species.

A further object of the present invention therefore relates to the use of a composition consisting of mixtures comprising a copper (II) salt of phosphorous acid and at least another metallic salt of phosphorous acid or the use of a composition consisting of mixtures based on a copper (II) salt of phosphorous acid, alone or mixed with at least another metallic salt of phosphorous acid and one or more fungicidal compounds, in particular one or more fungicidal compounds (1)-(34), for the control of phytopathogen fungi.

An object of the present invention also relates to a method for the control of phytopathogen fungi in agricultural crops by the application of a fungicidal composition consisting of mixtures comprising a copper (II) salt of phosphorous acid and at least another metallic salt of phosphorous acid or by the application of a composition consisting of mixtures based on a copper (II) salt of phosphorous acid alone or in a mixture with at least another metallic salt of phosphorous and one or more fungicidal compounds, in particular one or more fungicidal compounds (1)-(34).

Examples of pathogens controlled by the above compositions, together with examples of the application crops, are listed hereunder for purely illustrative and non-limiting purposes:

Plasmopara viticola (vines);
Phytophtora infestans (tomatoes, potatoes)
Phytophtora nicotianae (tobacco, ornamental plants);
Phytophtora palmivora (cacao);
Phytophtora cinnamomi (pineapples, citrus fruit);
Phytophtora capsici (peppers, tomatoes, cucurbits);
Phytophtora cryptogea (tomatoes, plums, ornamental plants);
Phytophtora megasperma (ornamental plants);
Phytophtora citri (citrus fruit);
Peronospora tabacina (tabacco);
Pseudoperonospora cubensis (cauliflowers, cucurbits);
Pseudoperonospora umili (hops);
Phytophtora cactorum (fruit trees);
Phytophtora destructor (onions);
Phytophtora fragarie (strawberries)
Bremia (salad).

The compositions, object of the present invention are capable of exerting a high synergistic fungicidal activity allowing preventive, protective, prophylactic, systemic, curative and de-rooting treatment to be effected.

In the above compositions, the copper (II) salt of phosphorous acid and the other metallic salts of phosphorous acid are preferably present in a ratio varying from 10/90 to 90/10 in equivalent phosphorous acid.

Even more preferably, the copper (II) salt of phosphorous acid and the other metallic salts of phosphorous acid are present in a ratio of 50/50 in equivalent phosphorous acid.

The compositions, object of the present invention, can be used in different quantities in relation to the crop, pathogen, environmental conditions and type of formulation adopted.

The fungicidal compositions according to the present invention generally envisage the following application dosages per hectare:

1,000-4,000 g of salt of phosphorous acid;
5-3,500 g for each fungicide from (1) to (34) present in the composition.

The application of the compositions, object of the present invention, can be effected on all parts of the plant, for example on the leaves, stems, branches and roots, or on the seeds themselves before sowing, or on the ground in which the plant grows.

The compositions according to the present invention can be used in agronomical practice in various forms, such as for example: dry powders, wettable powders, emulsifying concentrates, micro-emulsions, pastes, granulates, solutions, suspensions, etc. The choice of the type of composition will depend on the specific use.

The compositions are prepared in the known way, for example by diluting or dissolving the active substance with a solvent medium and/or a solid diluent, possibly in the presence of surface-active agents.

Solid diluents or supports which can be used are: silica, kaolin, bentonite, talc, infusorial earth, dolomite, calcium carbonate, magnesia, gypsum, clays, synthetic silicates, attapulgite, sepiolite.

Liquid diluents which can be used, are for example, in addition to water, aromatic compounds (xylols or blends of alkylbenzols), paraffins (petroleum fractions), alcohols (methanol, propanol, butanol, octanol, glycerine), amines, amides (N,N-dimethylformamide, Nmethylpyrrolidone), ketones (cyclohexanone, acetone, acetophenone, isophorone, ethylamylketone), fatty acids (for example, vegetable oils such as rape oil, sunflower oil), esters (isobutyl acetate, methyl esters of fatty acids obtained for example from the transesterification of vegetable oils).

Sodium salts, calcium salts, triethanolamine or triethylamine of alkylsulfonates, alkylaryl sulphonates, polyethoxylated alkylphenols, fatty alcohols condensed with ethylene oxide, polyethoxylated fatty acids, polyethoxylated esters of sorbitol, ligninsulphonates, can be used as surface-active agents.

The compositions can also contain special additives for particular purposes, such as for example, adhesive agents, such as gum Arabic, polyvinyl alcohol, polyvinyl pyrrolidone.

In the fungicidal compositions, object of the present invention, the concentration of active substances varies from 0.1% to 98% by weight, preferably from 0.5% to 90% by weight.

When desired, other compatible active principles can be added to the compositions, object of the present invention, such as, for example, phyto-regulators, antibiotics, herbicides, insecticides, fertilizers.

The following examples are provided for purely illustrative and non-limiting purposes of the present invention.

### EXAMPLE 1

### Preparation of the Copper and Calcium Salt of Phosphorous Acid (50:50)

### Mixture 1

350 g of H₃PO₃ and 350 ml of H₂O are charged into a 6 l flask. 563.5 g of KOH dissolved in 700 ml of H₂O and previously cooled to about 30° C. are added dropwise under stirring on an ice bath. After a few minutes, the aqueous solution obtained by dissolution of 364 g of CuCl₂*2H₂O and 313.95 g of CaCl₂*2H₂O in 3500 ml of H₂O is added dropwise. The blue-green precipitate which is formed is left under stirring for 24 hours at room temperature, the flask is then heated bringing the internal temperature to about 80° C. and the mixture is left under stirring for a further 4 hours approximately. The hot mixture is filtered on a Buchner funnel and the solid is washed with 3500 ml of H₂O previously heated to about 80° C. After drying in an oven, 652 g of the desired product are obtained.

Elemental analysis [% found (theoretical)]=Cu 20.9 (21.2); Ca 13.0 (13.4); P 20.5 (20.7).

### EXAMPLE 2

### Preparation of the Copper and Zinc Salt of Phosphorous Acid (50:50)

### Mixture 2

350 g of H₃PO₃ and 350 ml of H₂O are charged into a 6 l flask. 563.5 g of KOH dissolved in 700 ml of H₂O and previously cooled to about 30° C. are added dropwise under stirring on an ice bath. After a few minutes, the aqueous solution obtained by dissolution of 364 g of CuCl₂*2H₂O and 613.9 g of ZnSO₄*7H₂O in 3500 ml of H₂O is added dropwise. The blue precipitate which is formed is left under stirring for 24 hours at room temperature, the flask is then heated bringing the internal temperature to about 80° C. and the mixture is left under stirring for a further 4 hours approximately. The hot mixture is filtered on a Buchner funnel and the solid is washed with 3500 ml of H₂O previously heated to about 80° C. After drying in an oven, 699 g of the desired product are obtained.

Elemental analysis [% found (theoretical)]=Cu 18.6 (21.2); Zn 16.6 (13.4); P 18.4 (20.7); K 4.18 (0).

### EXAMPLE 3

### Preparation of the Copper and Manganese Salt of Phosphorous Acid (50:50)

### Mixture 3

350 g of H₃PO₃ and 350 ml of H₂O are charged into a 6 l flask. 563.5 g of KOH dissolved in 700 ml of H₂O and previously cooled to about 30° C. are added dropwise under stirring on an ice bath. After a few minutes, the aqueous solution obtained by dissolution of 364 g of CuCl₂*2H₂O and 360.86 g of MnSO₄*H₂O in 3500 ml of H₂O is added dropwise. The blue precipitate which is formed is left under stirring for 24 hours at room temperature, the flask is then heated bringing the internal temperature to about 80° C. and the mixture is left under stirring for a further 4 hours approximately. The hot mixture is filtered on a Buchner funnel and the solid is washed with 3500 ml of H₂O previously heated to about 80° C. After drying in an oven, 451.7 g of the desired product are obtained.

Elemental analysis [% found (theoretical)]=Cu 20.3 (21.4); Mn 16.8 (18.5); P 20.0 (20.9); K 1.49 (O).

### EXAMPLE 4

### Preparation of the Copper and Manganese Salt of Phosphorous Acid (66:33)

### Mixture 4

350 g of H₃PO₃ and 350 ml of H₂O are charged into a 6 l flask. 563.5 g of KOH dissolved in 700 ml of H₂O and previously cooled to about 30° C. are added dropwise under stirring on an ice bath. After a few minutes, the aqueous solution obtained by dissolution of 480.4 g of CuCl₂*2H₂O and 238.2 g of MnSO₄*H₂O in 3500 ml of H₂O is added dropwise. The blue precipitate which is formed is left under stirring for 24 hours at room temperature, the flask is then heated bringing the internal temperature to about 80° C. and the mixture is left under stirring for a further 4 hours approximately. The hot mixture is filtered on a Buchner funnel and the solid is washed with 3500 ml of H₂O previously heated to about 80° C. After drying in an oven, 482 g of the desired product are obtained.

Elemental analysis [% found (theoretical)]=Cu 27.7 (28.2); Mn 10.9 (12.2); P 18.0 (20.9); K 0.97 (0).

The following mixtures can be obtained with an analogous procedure:
Mixture 5: CuHPO₃+ZnHPO₃ (34:66)*
Mixture 6: CuHPO₃+ZnHPO₃ (66:34)*
Mixture 7: CuHPO₃+ZnHPO₃ (25:75)*
Mixture 8: CuHPO₃+ZnHPO₃ (75:25)*
Mixture 9: CuHPO₃+ZnHPO₃ (90:10)*
Mixture 10: CuHPO₃+ZnHPO₃ (10:90)*
Mixture 11: CuHPO₃+CaHPO₃ (34:66)*
Mixture 12: CuHPO₃+CaHPO₃ (66:34)*
Mixture 13: CuHPO₃+CaHPO₃ (25:75)*
Mixture 14: CuHPO₃+CaHPO₃ (75:25)*
Mixture 15: CuHPO₃+CaHPO₃ (90:10)*
Mixture 16: CuHPO₃+CaHPO₃ (10:90)*
Mixture 17: CuHPO₃+MnHPO₃ (34:66)*
Mixture 18: CuHPO₃+MnHPO₃ (25:75)*
Mixture 19: CuHPO₃+MnHPO₃ (75:25)*
Mixture 20: CuHPO₃+MnHPO₃ (90:10)*
Mixture 21: CuHPO₃+MnHPO₃ (10:90)*
Mixture 22: CuHPO₃+Al₂(HPO₃)₃ (50:50)*
Mixture 23: CuHPO₃+Al₂(HPO₃)₃ (34:66)*
Mixture 24: CuHPO₃+Al₂(HPO₃)₃ (66:34)*
Mixture 25: CuHPO₃+Al₂(HPO₃)₃ (25:75)*
Mixture 26: CuHPO₃+Al₂(HPO₃)₃ (75:25)*
Mixture 27: CuHPO₃+Al₂(HPO₃)₃ (90:10)*
Mixture 28: CuHPO₃+Al₂(HPO₃)₃ (10:90)*
Mixture 29: CuHPO₃+Fe₂(HPO₃)₃ (50:50)*
Mixture 30: CuHPO₃+Fe₂(HPO₃)₃ (34:66)*
Mixture 31: CuHPO₃+Fe₂(HPO₃)₃ (66:34)*
Mixture 32: CuHPO₃+Fe₂(HPO₃)₃ (25:75)*
Mixture 33: CuHPO₃+Fe₂(HPO₃)₃ (75:25)*
Mixture 34: CuHPO₃+Fe₂(HPO₃)₃ (90:10)*
Mixture 35: CuHPO₃+Fe₂(HPO₃)₃ (10:90)*
Mixture 36: CuHPO₃+NiHPO₃ (50:50)*
Mixture 37: CuHPO₃+NiHPO₃ (34:66)*
Mixture 38: CuHPO₃+NiHPO₃ (66:34)*
Mixture 39: CuHPO₃+NiHPO₃ (25:75)*
Mixture 40: CuHPO₃+NiHPO₃ (75:25)*
Mixture 41: CuHPO₃+NiHPO₃ (90:10)*
Mixture 42: CuHPO₃+NiHPO₃ (10:90)*
Mixture 43: CuHPO₃+MgHPO₃ (50:50)*
Mixture 44: CuHPO₃+MgHPO₃ (34:66)*
Mixture 45: CuHPO₃+MgHPO₃ (66:34)*
Mixture 46: CuHPO₃+MgHPO₃ (25:75)*
Mixture 47: CuHPO₃+MgHPO₃ (75:25)*
Mixture 48: CuHPO₃+MgHPO₃ (90:10)*
Mixture 49: CuHPO₃+MgHPO₃ (10:90)*
Mixture 50: CuHPO₃+BaHPO₃ (50:50)*
Mixture 51: CuHPO₃+BaHPO₃ (34:66)*
Mixture 52: CuHPO₃+BaHPO₃ (66:34)*
Mixture 53: CuHPO₃+BaHPO₃ (25:75)*
Mixture 54: CuHPO₃+BaHPO₃ (75:25)*
Mixture 55: CuHPO₃+BaHPO₃ (90:10)*
Mixture 56: CuHPO₃+BaHPO₃ (10:90)*
Mixture 57: CuHPO₃+K₂HPO₃ (50:50)*
Mixture 58: CuHPO₃+K₂HPO₃ (34:66)*
Mixture 59: CuHPO₃+K₂HPO₃ (66:34)*
Mixture 60: CuHPO₃+K₂HPO₃ (25:75)*
Mixture 61: CuHPO₃+K₂HPO₃ (75:25)*
Mixture 62: CuHPO₃+K₂HPO₃ (90:10)*
Mixture 63: CuHPO₃+K₂HPO₃ (10:90)*
Mixture 64: CuHPO₃+(NH₄)₂HPO₃ (50:50)*
Mixture 65: CuHPO₃+(NH₄)₂HPO₃ (34:66)*
Mixture 66: CuHPO₃+(NH₄)₂HPO₃ (66:34)*
Mixture 67: CuHPO₃+(NH₄)₂HPO₃ (25:75)*
Mixture 68: CuHPO₃+(NH₄)₂HPO₃ (75:25)*
Mixture 69: CuHPO₃+(NH₄)₂HPO₃ (90:10)*
Mixture 70: CuHPO₃+(NH₄)₂HPO₃ (10:90)*
Mixture 71: CuHPO₃+Ti(HPO₃)₂ (50:50)*
Mixture 72: CuHPO₃+Ti(HPO₃)₂ (34:66)*
Mixture 73: CuHPO₃+Ti(HPO₃)₂ (66:34)*
Mixture 74: CuHPO₃+Ti(HPO₃)₂ (25:75)*
Mixture 75: CuHPO₃+Ti(HPO₃)₂ (75:25)*
Mixture 76: CuHPO₃+Ti(HPO₃)₂ (90:10)*
Mixture 77: CuHPO₃+Ti(HPO₃)₂ (10:90)*
Mixture 78: CuHPO₃+Se(HPO₃)₂ (50:50)*
Mixture 79: CUHPO₃+Se(HPO₃)₂ (34:66)*
Mixture 80: CuHPO₃+Se(HPO₃)₂ (66:34)*
Mixture 81: CuHPO₃+Se(HPO₃)₂ (25:75)*
Mixture 82: CuHPO₃+Se(HPO₃)₂ (75:25)*
Mixture 83: CuHPO₃+Se(HPO₃)₂ (90:10)*
Mixture 84: CuHPO₃+Se(HPO₃)₂ (10:90)*
Mixture 85: CuHPO₃+CaHPO₃+MnHPO₃ (34:33:33)*
Mixture 86: CuHPO₃+CaHPO₃+MnHPO₃ (50:25:25)*
Mixture 87: CuHPO₃+CaHPO₃+ZnHPO₃ (34:33:33)*
Mixture 88: CuHPO₃+CaHPO₃+ZnHPO₃ (50:25:25)*
Mixture 89: CuHPO₃+ZnHPO₃+MnHPO₃ (34:33:33)*
Mixture 90: CuHPO₃+ZnHPO₃+MnHPO₃ (50:25:25)*
Mixture 91: CuHPO₃+CaHPO₃+K₂HPO₃ (34:33:33)*
Mixture 92: CuHPO₃+CaHPO₃+K₂HPO₃ (50:25:25)*
Mixture 93: CuHPO₃+CaHPO₃+K₂HPO₃ (25:25:50)*
Mixture 94: CuHPO₃+MnHPO₃+K₂HPO₃ (34:33:33)*
Mixture 95: CuHPO₃+MnHPO₃+K₂HPO₃ (50:25:25)*
Mixture 96: CuHPO₃+MnHPO₃+K₂HPO₃ (25:25:50)*
Mixture 97: CuHPO₃+ZnHPO₃+K₂HPO₃ (34:33:33)*
Mixture 98: CuHPO₃+ZnHPO₃+K₂HPO₃ (50:25:25)*
Mixture 99: CuHPO₃+ZnHPO₃+K₂HPO₃ (25:25:50)*
Mixture 100: Mixture 1+IR5885 (750⁽¹⁾:15⁽²⁾)
Mixture 101: Mixture 57+IR5885 (750⁽¹⁾:15⁽²⁾)
Mixture 102: Mixture 91+IR5885 (750⁽¹⁾:15⁽²⁾)
Mixture 103: Mixture 3+IR5885 (750⁽¹⁾:15⁽²⁾)
Mixture 104: Mixture 94+IR5885 (750⁽¹⁾:15⁽²⁾)
Mixture 105: Mixture 2+IR5885 (750⁽¹⁾:15⁽²⁾)
Mixture 106: Mixture 97+IR5885 (750⁽¹⁾:15⁽²⁾)
Mixture 107: Mixture 29+IR5885 (750⁽¹⁾:15⁽²⁾)
Mixture 108: CuHPO₃+Fe₂(HPO₃)₃+K₂HPO₃ (34:33:33)*
Mixture 109: Mixture 108+IR5885 (750⁽¹⁾:15⁽²⁾)
Mixture 110: Mixture 22+IR5885 (750⁽¹⁾:15⁽²⁾)
Mixture 111: CuHPO₃+Al₂(HPO₃)₃+K₂HPO₃ (34:33:33)*
Mixture 112: Mixture 111+IR5885 (750⁽¹⁾:15⁽²⁾)
Mixture 113: Mixture 64+IR5885 (750⁽¹⁾:15⁽²⁾)
Mixture 114: CuHPO₃+(NH₄)₂HPO₃+CaHPO₃ (34:33:33)*
Mixture 115: Mixture 114+IR5885 (750⁽¹⁾:15⁽²⁾)
Mixture 116: CuHPO₃+(NH₄)₂HPO₃+ZnHPO₃ (34:33:33)*
Mixture 117: Mixture 116+IR5885 (750⁽¹⁾:15⁽²⁾)
Mixture 118: CuHPO₃+(NH₄)₂HPO₃+MnHPO₃ (34:33:33)*
Mixture 119: Mixture 118+IR5885 (750⁽¹⁾:15⁽²⁾)
Mixture 120: CuHPO₃+(NH₄)₂HPO₃+Fe₂(HPO₃)₃ (34:33:33)*
Mixture 121: Mixture 120+IR5885 (750⁽¹⁾:15⁽²⁾)
Mixture 122: CuHPO₃+(NH₄)₂HPO₃+Al₂(HPO₃)₃ (34:33:33)*
Mixture 123: Mixture 122+IR5885 (750⁽¹⁾:15⁽²⁾)
Mixture 124: CuHPO₃+IR5885 (750⁽¹⁾:15⁽²⁾)
Mixture 125: CuHPO₃+IR6141 (750⁽¹⁾:15⁽²⁾)
Mixture 126: Mixture 1+IR6141 (750⁽¹⁾:15⁽²⁾)
Mixture 127: Mixture 57+IR6141 (750⁽¹⁾:15⁽²⁾)
Mixture 128: Mixture 91+IR6141 (750⁽¹⁾:15⁽²⁾)
Mixture 129: Mixture 3+IR6141 (750⁽¹⁾:15⁽²⁾)
Mixture 130: Mixture 94+IR6141 (750⁽¹⁾:15⁽²⁾)
Mixture 131: Mixture 2+IR6141 (750⁽¹⁾:15⁽²⁾)
Mixture 132: Mixture 97+IR6141 (750⁽¹⁾:15⁽²⁾)
Mixture 133: Mixture 29+IR6141 (750⁽¹⁾:15⁽²⁾)
Mixture 134: Mixture 108+IR6141 (750⁽¹⁾:15⁽²⁾)
Mixture 135: Mixture 22+IR6141 (750⁽¹⁾:15⁽²⁾)
Mixture 136: Mixture 111+IR6141 (750⁽¹⁾:15⁽²⁾)
Mixture 137: Mixture 64+IR6141 (750⁽¹⁾:15⁽²⁾)
Mixture 138: Mixture 114+IR6141 (750⁽¹⁾:15⁽²⁾)
Mixture 139: Mixture 116+IR6141 (750⁽¹⁾:15⁽²⁾)
Mixture 140: Mixture 118+IR6141 (750⁽¹⁾:15⁽²⁾)
Mixture 141: Mixture 120+IR6141 (750⁽¹⁾:15⁽²⁾)
Mixture 142: Mixture 122+IR6141 (750⁽¹⁾:15⁽²⁾)
Mixture 143: CuHPO₃+Cu(SA) (750⁽¹⁾:64⁽³⁾)
Mixture 144: Mixture 1+Cu(SA) (750⁽¹⁾:64⁽³⁾)
Mixture 145: Mixture 3+Cu(SA) (750⁽¹⁾:64⁽³⁾)
Mixture 146: Mixture 2+Cu(SA) (750⁽¹⁾:64⁽³⁾)
Mixture 147: Mixture 22+Cu(SA) (750⁽¹⁾:64⁽³⁾)
Mixture 148: Mixture 57+Cu(SA) (750⁽¹⁾:64⁽³⁾)
Mixture 149: Mixture 64+Cu(SA) (750⁽¹⁾:64⁽³⁾)
Mixture 150: Mixture 91+Cu(SA) (750⁽¹⁾:64⁽³⁾)
Mixture 151: Mixture 114+Cu(SA) (750⁽¹⁾:64⁽³⁾)
Mixture 152: CuHPO₃+Cu(ASA)₂ (750⁽¹⁾:64⁽³⁾)
Mixture 153: Mixture 1+Cu(ASA)₂ (750⁽¹⁾:64⁽³⁾)
Mixture 154: Mixture 3+Cu(ASA)₂ (750⁽¹⁾:64⁽³⁾)
Mixture 155: Mixture 2+Cu(ASA)₂ (750⁽¹⁾:64⁽³⁾)
Mixture 156: Mixture 22+Cu(ASA)₂ (750⁽¹⁾:64⁽³⁾)
Mixture 157: Mixture 57+Cu(ASA)₂ (750⁽¹⁾:64⁽³⁾)
Mixture 158: Mixture 64+Cu(ASA)₂ (750⁽¹⁾:64⁽³⁾)
Mixture 159: Mixture 91+Cu(ASA)₂ (750⁽¹⁾:64⁽³⁾)
Mixture 160: Mixture 114+Cu(ASA)₂ (750⁽¹⁾:64⁽³⁾)
Mixture 161: CuHPO₃+Benalaxyl (750⁽¹⁾:15⁽²⁾)
Mixture 162: Mixture 1+Benalaxyl (750⁽¹⁾:15⁽²⁾)
Mixture 163: Mixture 2+Benalaxyl (750⁽¹⁾:15⁽²⁾)
Mixture 164: Mixture 3+Benalaxyl (750⁽¹⁾:15⁽²⁾)
Mixture 165: Mixture 22+Benalaxyl (750⁽¹⁾:15⁽²⁾)
Mixture 166: Mixture 57+Benalaxyl (750⁽¹⁾:15⁽²⁾)
Mixture 167: Mixture 64+Benalaxyl (750⁽¹⁾:15⁽²⁾)
Mixture 168: Mixture 91+Benalaxyl (750⁽¹⁾:15⁽²⁾)
Mixture 169: Mixture 114+Benalaxyl (750⁽¹⁾:15⁽²⁾)
Mixture 170: CuHPO₃+Metalaxyl (750⁽¹⁾:15⁽²⁾)
Mixture 171: Mixture 1+Metalaxyl (750⁽¹⁾:15⁽²⁾)
Mixture 172: Mixture 2+Metalaxyl (750⁽¹⁾:15⁽²⁾)
Mixture 173: Mixture 3+Metalaxyl (750⁽¹⁾:15⁽²⁾)
Mixture 174: Mixture 22+Metalaxyl (750⁽¹⁾:15⁽²⁾)
Mixture 175: Mixture 57+Metalaxyl (750⁽¹⁾:15⁽²⁾)
Mixture 176: Mixture 64+Metalaxyl (750⁽¹⁾:15⁽²⁾)
Mixture 177: Mixture 91+Metalaxyl (750⁽¹⁾:15⁽²⁾)
Mixture 178: Mixture 114+Metalaxyl (750⁽¹⁾:15⁽²⁾)
Mixture 179: CuHPO₃+Metalaxyl-M (750⁽¹⁾:15⁽²⁾)
Mixture 180: Mixture 1+Metalaxyl-M (750⁽¹⁾:15⁽²⁾)
Mixture 181: Mixture 2+Metalaxyl-M (750⁽¹⁾:15⁽²⁾)
Mixture 182: Mixture 3+Metalaxyl-M (750⁽¹⁾:15⁽²⁾)
Mixture 183: Mixture 22+Metalaxyl-M (750⁽¹⁾:15⁽²⁾)
Mixture 184: Mixture 57+Metalaxyl-M (750⁽¹⁾:15⁽²⁾)
Mixture 185: Mixture 64+Metalaxyl-M (750⁽¹⁾:15⁽²⁾)
Mixture 186: Mixture 91+Metalaxyl-M (750⁽¹⁾:15⁽²⁾)
Mixture 187: Mixture 114+Metalaxyl-M (750⁽¹⁾:15⁽²⁾)
Mixture 188: CuHPO₃+Iprovalicarb (750⁽¹⁾:15⁽²⁾)
Mixture 189: Mixture 1+Iprovalicarb (750⁽¹⁾:15⁽²⁾)
Mixture 190: Mixture 2+Iprovalicarb (750⁽¹⁾:15⁽²⁾)
Mixture 191: Mixture 3+Iprovalicarb (750⁽¹⁾:15⁽²⁾)
Mixture 192 Mixture 22+Iprovalicarb (750⁽¹⁾:15⁽²⁾)
Mixture 193: Mixture 57+Iprovalicarb (750⁽¹⁾:15²)
Mixture 194: Mixture 64+Iprovalicarb (750⁽¹⁾:15⁽²⁾)
Mixture 195: Mixture 91+Iprovalicarb (750⁽¹⁾:15⁽²⁾)
Mixture 196: Mixture 114+Iprovalicarb (750⁽¹⁾:15⁽²⁾)
Mixture 197: CuHPO₃+Axoxystrobin (750⁽¹⁾:10⁽²⁾)
Mixture 198: Mixture 1+Axoxystrobin (750⁽¹⁾:10⁽²⁾)
Mixture 199: Mixture 2+Axoxystrobin (750⁽¹⁾:10⁽²⁾)
Mixture 200: Mixture 3+Axoxystrobin (750⁽¹⁾:10⁽²⁾)
Mixture 201: Mixture 22+Axoxystrobin (750⁽¹⁾:10⁽²⁾)
Mixture 202: Mixture 57+Axoxystrobin (750⁽¹⁾:10⁽²⁾)
Mixture 203: Mixture 64+Axoxystrobin (750⁽¹⁾:10⁽²⁾)
Mixture 204: Mixture 91+Axoxystrobin (750⁽¹⁾:10⁽²⁾)
Mixture 205: Mixture 114+Axoxystrobin (750⁽¹⁾:10⁽²⁾)
Mixture 206: CuHPO₃+Metominofen (750⁽¹⁾:10⁽²⁾)
Mixture 207: Mixture 1+Metominofen (750⁽¹⁾:10⁽²⁾)
Mixture 208: Mixture 2+Metominofen (750⁽¹⁾:10⁽²⁾)
Mixture 209: Mixture 3+Metominofen (750⁽¹⁾:10⁽²⁾)
Mixture 210: Mixture 22+Metominofen (750⁽¹⁾:10⁽²⁾)
Mixture 211: Mixture 57+Metominofen (750⁽¹⁾:10⁽²⁾)
Mixture 212: Mixture 64+Metominofen (750⁽¹⁾:10⁽²⁾)
Mixture 213: Mixture 91+Metominofen (750⁽¹⁾:10⁽²⁾)
Mixture 214: Mixture 114+Metominofen (750⁽¹⁾:10⁽²⁾)
Mixture 215: CuHPO₃+Acibenzolar (750⁽¹⁾:20⁽²⁾)
Mixture 216: Mixture 1+Acibenzolar (750⁽¹⁾:20⁽²⁾)
Mixture 217: Mixture 2+Acibenzolar (750⁽¹⁾:20⁽²⁾)
Mixture 218: Mixture 3+Acibenzolar (750⁽¹⁾:20⁽²⁾)
Mixture 219: Mixture 22+Acibenzolar (750⁽¹⁾:20⁽²⁾)
Mixture 220: Mixture 57+Acibenzolar (750⁽¹⁾:20⁽²⁾)
Mixture 221: Mixture 64+Acibenzolar (750⁽¹⁾:20⁽²⁾)
Mixture 222: Mixture 91+Acibenzolar (750⁽¹⁾:20⁽²⁾)
Mixture 223: Mixture 114+Acibenzolar (750⁽¹⁾:20⁽²⁾)
Mixture 224: CuHPO₃+Cyazofamide (750⁽¹⁾:10⁽²⁾)
Mixture 225: Mixture 1+Cyazofamide (750⁽¹⁾:10⁽²⁾)
Mixture 226: Mixture 2+Cyazofamide (750⁽¹⁾:10⁽²⁾
Mixture 227: Mixture 3+Cyazofamide (750⁽¹⁾:10⁽²⁾)
Mixture 228: Mixture 22+Cyazofamide (750⁽¹⁾:10⁽²⁾)
Mixture 229: Mixture 57+Cyazofamide (750⁽¹⁾:10⁽²⁾)
Mixture 230: Mixture 64+Cyazofamide (750⁽¹⁾:10⁽²⁾)
Mixture 231: Mixture 91+Cyazofamide (750⁽¹⁾:10⁽²⁾)
Mixture 232: Mixture 114+Cyazofamide (750⁽¹⁾:20⁽²⁾)
Mixture 233: CuHPO₃+Chlorothalonil (750⁽¹⁾:100⁽²⁾)
Mixture 234: Mixture 1+Chlorothalonil (750⁽¹⁾:100⁽²⁾)
Mixture 235: Mixture 2+Chlorothalonil (750⁽¹⁾:100⁽²⁾)
Mixture 236: Mixture 3+Chlorothalonil (750⁽¹⁾:100⁽²⁾)
Mixture 237: Mixture 22+Chlorothalonil (750⁽¹⁾:100⁽²⁾)
Mixture 238: Mixture 57+Chlorothalonil (750⁽¹⁾:100⁽²⁾)
Mixture 239: Mixture 64+Chlorothalonil (750⁽¹⁾:100⁽²⁾)
Mixture 240: Mixture 91+Chlorothalonil (750⁽¹⁾:100⁽²⁾)
Mixture 241: Mixture 114+Chlorothalonil (750⁽¹⁾:100⁽²⁾)
Mixture 242: CuHPO₃+Bordeaux mixt. (750⁽¹⁾:200⁽³⁾)
Mixture 243: Mixture 1+Bordeaux mixt. (750⁽¹⁾:200⁽³⁾)
Mixture 244: Mixture 2+Bordeaux mixt. (750⁽¹⁾:200⁽³⁾)
Mixture 245: Mixture 3+Bordeaux mixt. (750⁽¹⁾:200⁽³⁾)
Mixture 246: Mixture 22+Bordeaux mixt. (750⁽¹⁾:200⁽³⁾)
Mixture 247: Mixture 57+Bordeaux mixt. (750⁽¹⁾:200⁽³⁾)
Mixture 248: Mixture 64+Bordeaux mixt. (750⁽¹⁾:200⁽³⁾)
Mixture 249: Mixture 91+Bordeaux mixt. (750⁽¹⁾:200⁽³⁾)
Mixture 250: Mixture 114+Bordeaux mixt. (750⁽¹⁾:200⁽³⁾)
Mixture 251: CuHPO₃+Cu(OH)₂ (750⁽¹⁾:100⁽³⁾)
Mixture 252: Mixture 1+Cu(OH)₂ (750⁽¹⁾:100⁽³⁾)
Mixture 253: Mixture 2+Cu(OH)₂ (750⁽¹⁾:100⁽³⁾)
Mixture 254: Mixture 3+Cu(OH)₂ (750⁽¹⁾:100⁽³⁾)
Mixture 255: Mixture 22+Cu(OH)₂ (750⁽¹⁾:100⁽³⁾)
Mixture 256: Mixture 57+Cu(OH)₂ (750⁽¹⁾:100⁽³⁾)
Mixture 257: Mixture 64+Cu(OH)₂ (750⁽¹⁾:100⁽³⁾)
Mixture 258: Mixture 91+Cu(OH)₂ (750⁽¹⁾:100⁽³⁾)
Mixture 259: Mixture 114+Cu(OH)₂ (750⁽¹⁾:100⁽³⁾)
Mixture 260: CuHPO₃+CuSO₄ (750⁽¹⁾:100⁽³⁾)
Mixture 261: Mixture 1+CuSO₄ (750⁽¹⁾:100⁽³⁾)
Mixture 262: Mixture 2+CuSO₄ (750⁽¹⁾:100⁽³⁾)
Mixture 263: Mixture 3+CuSO₄ (750⁽¹⁾:100⁽³⁾)
Mixture 264: Mixture 22+CuSO₄ (750⁽¹⁾:100⁽³⁾)
Mixture 265: Mixture 57+CuSO₄ (750⁽¹⁾:100⁽³⁾)
Mixture 266: Mixture 64+CuSO₄ (750⁽¹⁾:100⁽³⁾)
Mixture 267: Mixture 91+CuSO₄ (750⁽¹⁾:100⁽³⁾)
Mixture 268: Mixture 114+CuSO₄ (750⁽¹⁾:100⁽³⁾)
Mixture 269: CuHPO₃+CuCl₂*3Cu(OH)₂ (750⁽¹⁾:200⁽³⁾)
Mixture 270: Mixture 1+CuCl₂*3Cu(OH)₂ (750⁽¹⁾:200⁽³⁾)
Mixture 271: Mixture 2+CuCl₂*3Cu(OH)₂ (750⁽¹⁾:200⁽³⁾)
Mixture 272: Mixture 3+CuCl₂*3Cu(OH)₂ (750⁽¹⁾:200⁽³⁾)
Mixture 273: Mixture 22+CuCl₂*3Cu(OH)₂ (750⁽¹⁾:200⁽³⁾)
Mixture 274: Mixture 57+CuCl₂*3Cu(OH)₂ (750⁽¹⁾:200⁽³⁾)
Mixture 275: Mixture 64+CuCl₂*3Cu(OH)₂ (750⁽¹⁾:200⁽³⁾)
Mixture 276: Mixture 91+CUCl₂*3Cu(OH)₂ (750⁽¹⁾:200⁽³⁾)
Mixture 277: Mixture 114+CuCl₂*3Cu(OH)₂ (750⁽¹⁾:200⁽³⁾)
Mixture 278: CuHPO₃+Airone (750⁽¹⁾:40⁽³⁾)
Mixture 279: Mixture 1+Airone (750⁽¹⁾:40⁽³⁾)
Mixture 280: Mixture 2+Airone (750⁽¹⁾:40⁽³⁾)
Mixture 281: Mixture 3+Airone (750⁽¹⁾:40⁽³⁾)
Mixture 282: Mixture 22+Airone (750⁽¹⁾:40⁽³⁾)
Mixture 283: Mixture 57+Airone (750⁽¹⁾:40⁽³⁾)
Mixture 284: Mixture 64+Airone (750⁽¹⁾:40⁽³⁾)
Mixture 285: Mixture 91+Airone (750⁽¹⁾:40⁽³⁾)
Mixture 286: Mixture 114+Airone (750⁽¹⁾:40⁽³⁾)
Mixture 287: CuHPO₃+Pyraclostrobin (750⁽¹⁾:10⁽²⁾)
Mixture 288: Mixture 1+Pyraclostrobin (750⁽¹⁾:10⁽²⁾)
Mixture 289: Mixture 2+Pyraclostrobin (750⁽¹⁾:10⁽²⁾)
Mixture 290: Mixture 3+Pyraclostrobin (750⁽¹⁾:10⁽²⁾)
Mixture 291: Mixture 22+Pyraclostrobin (750⁽¹⁾:10⁽²⁾)
Mixture 292: Mixture 57+Pyraclostrobin (750⁽¹⁾:10⁽²⁾)
Mixture 293: Mixture 64+Pyraclostrobin (750⁽¹⁾:10⁽²⁾)
Mixture 294: Mixture 91+Pyraclostrobin (750⁽¹⁾:10⁽²⁾)
Mixture 295: Mixture 114+Pyraclostrobin (750⁽¹⁾:10⁽²⁾)
Mixture 296: CuHPO₃+Benthiavalicarb (750⁽¹⁾:15⁽²⁾)
Mixture 297: Mixture 1+Benthiavalicarb (750⁽¹⁾:15⁽²⁾)
Mixture 298: Mixture 2+Benthiavalicarb (750⁽¹⁾:15⁽²⁾)
Mixture 299: Mixture 3+Benthiavalicarb (750⁽¹⁾:15⁽²⁾)
Mixture 300: Mixture 22+Benthiavalicarb (750⁽¹⁾:15⁽²⁾)
Mixture 301: Mixture 57+Benthiavalicarb (750⁽¹⁾:15⁽²⁾)
Mixture 302: Mixture 64+Benthiavalicarb (750⁽¹⁾:15⁽²⁾)
Mixture 303: Mixture 91+Benthiavalicarb (750⁽¹⁾:15⁽²⁾)
Mixture 304: Mixture 114+Benthiavalicarb (750⁽¹⁾:15⁽²⁾)
Mixture 305: CuHPO₃+Cymoxanil (750⁽¹⁾:100⁽²⁾)
Mixture 306: Mixture 1+Cymoxanil (750⁽¹⁾:100⁽²⁾)
Mixture 307: Mixture 2+Cymoxanil (750⁽¹⁾:100⁽²⁾)
Mixture 308: Mixture 3+Cymoxanil (750⁽¹⁾:100⁽²⁾)
Mixture 309: CuHPO₃+Oxadixyl (750⁽¹⁾:15⁽²⁾)
Mixture 310: Mixture 1+Oxadixyl (750⁽¹⁾:15⁽²⁾)
Mixture 311: Mixture 2+Oxadixyl (750⁽¹⁾:15⁽²⁾)
Mixture 312: Mixture 3+Oxadixyl (750⁽¹⁾:15⁽²⁾)
Mixture 313: CuHPO₃+Ofurace (750⁽¹⁾:15⁽²⁾)
Mixture 314: Mixture 1+Ofurace (750⁽¹⁾:15⁽²⁾)
Mixture 315: Mixture 2+Ofurace (750⁽¹⁾:15⁽²⁾)
Mixture 316: Mixture 3+Ofurace (750⁽¹⁾:15⁽²⁾)
Mixture 317: CuHPO₃+Famoxadone (750⁽¹⁾:100⁽²⁾)
Mixture 318: Mixture 1+Famoxadone (750⁽¹⁾:100⁽²⁾)
Mixture 319: Mixture 2+Famoxadone (750⁽¹⁾:100⁽²⁾)
Mixture 320: Mixture 3+Famoxadone (750⁽¹⁾:100⁽²⁾)
Mixture 321: CuHPO₃+Fenamidone (750⁽¹⁾:50⁽²⁾)
Mixture 322: Mixture 1+Fenamidone (750⁽¹⁾:50⁽²⁾)
Mixture 323: Mixture 2+Fenamidone (750⁽¹⁾:50⁽²⁾)
Mixture 324: Mixture 3+Fenamidone (750⁽¹⁾:50⁽²⁾)
Mixture 325: CuHPO₃+Fluazinam (750⁽¹⁾:100⁽²⁾)
Mixture 326: Mixture 1+Fluazinam (750⁽¹⁾:100⁽²⁾)
Mixture 327: Mixture 2+Fluazinam (750⁽¹⁾:100⁽²⁾)
Mixture 328: Mixture 3+Fluazinam (750⁽¹⁾:100⁽²⁾)
Mixture 329: CuHPO₃+Dimethomorph (750⁽¹⁾:100⁽²⁾)
Mixture 330: Mixture 1+Dimethomorph (750⁽¹⁾:100⁽²⁾)
Mixture 331: Mixture 2+Dimethomorph (750⁽¹⁾:100⁽²⁾)
Mixture 332: Mixture 3+Dimethomorph (750⁽¹⁾:100⁽²⁾)
Mixture 333: CuHPO₃+Mancozeb (750⁽¹⁾:200⁽²⁾)
Mixture 334: Mixture 1+Mancozeb (750⁽¹⁾:200⁽²⁾)
Mixture 335: Mixture 2+Mancozeb (750⁽¹⁾:200⁽²⁾)
Mixture 336: Mixture 3+Mancozeb (750⁽¹⁾:200⁽²⁾)
Mixture 337: CuHPO₃+Tolylfluanide (750⁽¹⁾:100⁽²⁾)
Mixture 338: Mixture 1+Tolylfluanide (750⁽¹⁾:100⁽²⁾)
Mixture 339: Mixture 2+Tolylfluanide (750⁽¹⁾:100⁽²⁾)
Mixture 340: Mixture 3+Tolylfluanide (750⁽¹⁾:100⁽²⁾)
Mixture 341: CuHPO₃+Folpet (750⁽¹⁾:100⁽²⁾)
Mixture 342: Mixture 1+Folpet (750⁽¹⁾:100⁽²⁾)
Mixture 343: Mixture 2+Folpet (750⁽¹⁾:100⁽²⁾)
Mixture 344: Mixture 3+Folpet (750⁽¹⁾:100⁽²⁾)
Mixture 345: CuHPO₃+Propamocarb (750⁽¹⁾:200⁽²⁾)
Mixture 346: Mixture 1+Propamocarb (750⁽¹⁾:200⁽²⁾)
Mixture 347: Mixture 2+Propamocarb (750⁽¹⁾:200⁽²⁾)
Mixture 348: Mixture 3+Propamocarb (750⁽¹⁾:200⁽²⁾)
Mixture 349: CuHPO₃+Zoxamide (750⁽¹⁾:100⁽²⁾)
Mixture 350: Mixture 1+Zoxamide (750⁽¹⁾:100⁽²⁾)
Mixture 351: Mixture 2+Zoxamide (750⁽¹⁾:100⁽²⁾)
Mixture 352: Mixture 3+Zoxamide (750⁽¹⁾:100⁽²⁾)
Mixture 353: CuHPO₃+Syngenta⁽⁴⁾(750⁽¹⁾:15⁽²⁾)
Mixture 354: Mixture 1+Syngenta⁽⁴⁾(750⁽¹⁾:15⁽²⁾)
Mixture 355: Mixture 2+Syngenta⁽⁴⁾(750⁽¹⁾:15⁽²⁾)
Mixture 356: Mixture 3+Syngenta⁽⁴⁾(750⁽¹⁾:15⁽²⁾)
Mixture 357: CuHPO₃+Ethaboxam (750⁽¹⁾:50⁽²⁾)
Mixture 358: Mixture 1+Ethaboxam (750⁽¹⁾:50⁽²⁾)
Mixture 359: Mixture 2+Ethaboxam (750⁽¹⁾:50⁽²⁾)
Mixture 360: Mixture 3+Ethaboxam (750⁽¹⁾:50⁽²⁾)
Mixture 361: CuHPO₃+Saccharin (750⁽¹⁾:125⁽²⁾)
Mixture 362: Mixture 1+Saccharin (750⁽¹⁾:125⁽²⁾)
Mixture 363: Mixture 2+Saccharin (750⁽¹⁾:125⁽²⁾)
Mixture 364: Mixture 3+Saccharin (750⁽¹⁾:125⁽²⁾)
*: reciprocal proportions expressed in equivalent phosphorous acid.
   (1): dose expressed in ppm of equivalent phosphorous acid.
   (2): dose expressed in ppm of active ingredient.
   (3): dose expressed in ppm of equivalent metal copper.
   (4): fungicidal compound (32).

### EXAMPLE 5

### Efficacy of the Fungicidal Compositions, Object of the Present Invention, in the Control of Plasmopara viticola on Vines in Preventive Leaf Application

### Tables 1-2

Leaves of cultivar Merlot vines, grown in vases, in a conditioned environment (20±1° C., 70% relative humidity) are treated by spraying both sides of the leaves with the mixtures under examination dispersed in an aqueous solution containing a 0.3% of tween 20.

After remaining 7 days in a conditioned environment, the plants are infected on the lower side with an aqueous suspension of spores of Plasmopara viticola (200,000 spores per cm³).

The plants are kept in a humidity saturated environment, at 21° C., for the incubation period of the fungus and, at the end of this period (7 days), the fungicidal activity is evaluated according to an evaluation percentage scale from 100 (healthy plant) to 0 (completely infected plant).

From the data indicated in tables 1-2, it is possible to verify the synergistic effect of the mixtures, consisting of the mixtures being examined, compared with the expected efficacy using the Limpel formula (“Pesticide Science” (1987), vol. 19, pages 309-315):

E=x+y−(xy/100)

wherein:
E is the fungicidal activity expected, in the absence of synergistic effects, from a mixture obtained by mixing g·x of compound X with g·y of compound Y;
x is the activity of compound X when used alone with a dosage of g·x;
y is the activity of compound Y when used alone with a dosage of g·y.

When the fungicidal activity found experimentally is greater than the value of E, this activity is considered synergistic.

TABLE 1
7-day preventive activity on Plasmopara viticola, with
leaf application on vines, of mixtures (50:50)* of copper
(II) salt of phosphorous acid (CuHPO₃), which at 500 ppm**
(g.x) is 88 (x) with other metallic salts of phosphorous acid.

| | | | Activity | | |
|---|---|---|---|---|---|
| | | | Mixture | | |
| | Dose | | according | Experimental | |
| | ppm** | Activity | to Limpel | activity of | Synergy |
| Mixture | (g.y) | (y) | (E) | mixture | factor |
| CaHPO₃ | 500 | 0 | 88 | 100 | 1.14 |
| ZnHPO₃ | 500 | 25 | 91 | 100 | 1.10 |
| MnHPO₃ | 500 | 0 | 88 | 98 | 1.11 |
| (NH₄)₂HPO₃ | 500 | 38 | 92.5 | 98 | 1.06 |
| K₂HPO₃ | 500 | 45 | 93.4 | 99 | 1.06 |

*reciprocal proportions expressed in equivalent phosphorous acid.
**the doses in ppm refer to the quantity of equivalent phosphorous acid.

TABLE 2
7-day preventive activity on Plasmopara viticola, with
leaf application on vines, of mixtures of the Saccharin
compound (compound Nr. 34), which at 500 ppm (g.x) is 45
(x) with one or more metallic salts of phosphorous acid.

| | | | Activity | | |
|---|---|---|---|---|---|
| | | | Mixture | | |
| | Dose | | according | Experimental | |
| | ppm** | Activity | to Limpel | activity of | Synergy |
| Mixture | (g.y) | (y) | (E) | mixture | factor |
| CuHPO₃ | 250 | 35 | 64.2 | 100 | 1.56 |
| CuHPO₃ + | 250 | 50 | 72.5 | 100 | 1.38 |
| CaHPO₃ | | | | | |
| (50:50)* | | | | | |
| CuHPO₃ + | 250 | 52 | 73.6 | 98 | 1.33 |
| ZnHPO₃ | | | | | |
| (50:50)* | | | | | |
| CuHPO₃ + | 250 | 47 | 70.8 | 95 | 1.34 |
| MnHPO₃ | | | | | |
| (50:50)* | | | | | |
| CuHPO₃ + | 250 | 56 | 75.8 | 98 | 1.29 |
| MnHPO₃ | | | | | |
| (66:34)* | | | | | |

*reciprocal proportions expressed in equivalent phosphorous acid.
**the doses in ppm refer to the quantity of equivalent phosphorous acid.

### EXAMPLE 6

### Efficacy of the Fungicidal Compositions, Object of the Present Invention, in the Control of Phytophthora infestans on Potato Plants in Preventive Leaf Application

### Tables 3-6

Leaves of potato plants, of the Primura variety, grown in vases, in a conditioned environment (20±1° C., 70% relative humidity) are treated by spraying both sides of the leaves with the mixtures under examination dispersed in an aqueous solution containing 0.3% of tween 20.

After remaining 7 days in a conditioned environment, the plants are infected on the lower side with an aqueous suspension of spores of Phytophthora infestans (100,000 spores per cm³).

The plants are kept in a humidity saturated environment, at 18-24° C., for the incubation period of the fungus and, at the end of this period (7 days), the fungicidal activity is evaluated according to an evaluation percentage scale from 100 (healthy plant) to 0 (completely infected plant).

From the data indicated in tables 3-6, it is possible to verify the synergic effect of the mixtures, consisting of the mixtures being examined, compared with the expected efficacy using the Limpel formula.

TABLE 3
7-day preventive activity on Phytophthora infestans, with
leaf application on potato plants, of mixtures (50:50)*
of copper (II) salt of phosphorous acid (CuHPO₃), which
at 750 ppm** (g.x) is 12 (x) with other metallic salts of
phosphorous acid.

| | | | Activity | | |
|---|---|---|---|---|---|
| | | | Mixture | | |
| | Dose | | according | Experimental | |
| | ppm** | Activity | to Limpel | activity of | Synergy |
| Mixture | (g.y) | (y) | (E) | mixture | factor |
| CaHPO₃ | 750 | 10 | 18.8 | 78 | 4.1 |
| ZnHPO₃ | 750 | 5 | 16.4 | 70 | 4.2 |
| MnHPO₃ | 750 | 15 | 25.2 | 80 | 3.2 |
| (NH₄)₂HPO₃ | 750 | 5 | 16.4 | 68 | 4.1 |
| K₂HPO₃ | 750 | 5 | 16.4 | 65 | 3.9 |

*reciprocal proportions expressed in equivalent phosphorous acid.
**the doses in ppm refer to the quantity of equivalent phosphorous acid.

TABLE 4
7-day preventive activity on Phytophthora infestans, with
leaf application on potato plants, of mixtures of the
compound IR5885(compound Nr. 1), which at 15 ppm (g.x) is
40 (x) with one or more metallic salts of phosphorous
acid.

| | | | Activity | | |
|---|---|---|---|---|---|
| | | | Mixture | | |
| | Dose | | according | Experimental | |
| | ppm** | Activity | to Limpel | activity of | Synergy |
| Mixture | (g.y) | (y) | (E) | mixture | factor |
| CuHPO₃ | 750 | 12 | 47.2 | 95 | 2.01 |
| CuHPO₃ + | 750 | 78 | 86.8 | 95 | 1.09 |
| CaHPO₃ | | | | | |
| (50:50)* | | | | | |
| CuHPO₃ + | 750 | 70 | 82 | 90 | 1.1 |
| ZnHPO₃ | | | | | |
| (50:50)* | | | | | |
| CuHPO₃ + | 750 | 80 | 88 | 93 | 1.05 |
| MnHPO₃ | | | | | |
| (50:50)* | | | | | |
| CuHPO₃ + | 750 | 75 | 78.7 | 90 | 1.14 |
| MnHPO₃ | | | | | |
| (66:34)* | | | | | |

*reciprocal proportions expressed in equivalent phosphorous acid.
**the doses in ppm refer to the quantity of equivalent phosphorous acid.

TABLE 5
7-day preventive activity on Phytophthora infestans, with
leaf application on potato plants, of mixtures of the
compound IR6141(compound Nr. 2), which at 50 ppm (g.x) is
35 (x) with one or more metallic salts of phosphorous acid.

| | | | Activity | | |
|---|---|---|---|---|---|
| | | | Mixture | | |
| | Dose | | according | Experimental | |
| | ppm** | Activity | to Limpel | activity of | Synergy |
| Mixture | (g.y) | (y) | (E) | mixture | factor |
| CuHPO₃ | 750 | 12 | 42.8 | 90 | 2.10 |
| CuHPO₃ + | 750 | 78 | 85.7 | 94 | 1.09 |
| CaHPO₃ | | | | | |
| (50:50)* | | | | | |
| CuHPO₃ + | 750 | 70 | 80.5 | 93 | 1.15 |
| ZnHPO₃ | | | | | |
| (50:50)* | | | | | |
| CuHPO₃ + | 750 | 80 | 87 | 96 | 1.10 |
| MnHPO₃ | | | | | |
| (50:50)* | | | | | |
| CuHPO₃ + | 750 | 77 | 85 | 94 | 1.1 |
| MnHPO₃ | | | | | |
| (66:34)* | | | | | |

*reciprocal proportions expressed in equivalent phosphorous acid.
**the doses in ppm refer to the quantity of equivalent phosphorous acid.

acid.

TABLE 6
7-day preventive activity on Phytophthora infestans,
strain resistant to phenylalanine, with leaf application
on potato plants, of mixtures of the compound
IR6141(compound Nr. 2), which at 50 ppm (g.x) is 5 (x)
with one or more metallic salts of phosphorous acid.

| | | | Activity | | |
|---|---|---|---|---|---|
| | | | Mixture | | |
| | Dose | | according | Experimental | |
| | ppm** | Activity | to Limpel | activity of | Synergy |
| Mixture | (g.y) | (y) | (E) | mixture | factor |
| CuHPO₃ | 750 | 12 | 16.4 | 85 | 5.2 |
| CuHPO₃ + | 750 | 78 | 79.1 | 90 | 1.14 |
| CaHPO₃ | | | | | |
| (50:50)* | | | | | |
| CuHPO₃ + | 750 | 70 | 71.5 | 88 | 1.23 |
| ZnHPO₃ | | | | | |
| (50:50)* | | | | | |
| CuHPO₃ + | 750 | 80 | 81 | 94 | 1.16 |
| MnHPO₃ | | | | | |
| (50:50)* | | | | | |
| CuHPO₃ + | 750 | 77 | 78.1 | 90 | 1.15 |
| MnHPO₃ | | | | | |
| (66:34)* | | | | | |

*reciprocal proportions expressed in equivalent phosphorous acid.
**the doses in ppm refer to the quantity of equivalent phosphorous acid.

1. A fungicidal composition consisting of mixtures comprising a copper (II) salt of phosphorous acid and at least another metallic salt of phosphorous acid or consisting of mixtures comprising a copper (II) salt of phosphorous acid, alone or mixed with at least another metallic salt of phosphorous acid, and one or more fungicidal compounds.

2. The fungicidal composition according to claim 1, characterized in that the metallic salt of phosphorous acid is a salt of an alkaline, alkaline earth metal, an ammonium salt or a salt of Fe, Mn, Zn, Ni, Al, Ti or Se.

3. The fungicidal composition according to claim 1, characterized in that the metallic salt of phosphorous acid is a mono- or di-basic salt, or a mixture of these in any proportion.

4. The fungicidal composition according to claim 1, characterized in that the mixtures also contain alkaline metals in percentages not higher than 10% by weight.

5. The fungicidal composition according to claim 1,
characterized in that the fungicidal compound is selected from:
(1) IR5885, a dipeptide compound corresponding to diastereoisomeric mixtures of methyl [S—(R,S)]-[3-(N-isopropoxycarbonylvalinyl)-amino]-3-(4-chloro-phenyl) propanoate in any proportion, or to one of the two diastereoisomeric forms S—R or S—S taken individually;
(2) IR6141, corresponding to N-(phenylacetyl)-N-2,6-xylyl-R-methyl alaninate;
(3) Salicylic acid (SA) or its derivatives such as acetylsalicylic acid (ASA), copper salts of salicylic acid or acetylsalicylic acid;
(4) A copper (I) or copper (II) salt, such as copper oxychloride, copper hydroxide, Bordeaux mixture, copper sulfate, or a mixture of copper hydroxide and oxychloride (Airone);
(5) Benalaxyl corresponding to N-(phenylacetyl)-N-2,6-xylyl-RS-methyl alaninate; (6) Metalaxyl corresponding to N-(2-methoxyacetyl)-N-2,6-xylyl-RS-methyl alaninate;
(7) Metalaxyl-M corresponding to N-(2-methoxyacetyl)-N-2,6-xylyl-R-methyl alaninate;
(8) Oxadixyl corresponding to 2-methoxy-N-(2-oxo-1,3-oxazolidin-3-yl)aceto-2′ 6-xylidide;
(9) Ofurace corresponding to DL-3-[N-chloroacetyl-N-(2,6-xylyl)-amino]-γ-butyrolactone;
(10) Iprovalicarb corresponding to 0-(1-methyl-ethyl)-N-[2-methyl-1-[[[1-(4-methyl-phenyl)-ethyl]amino]carbonyl]propyl]carbamate;
(11) Benthiavalicarb-isopropyl corresponding to O— isopropyl [(S)-1-{[(1R)-1-(6-fluoro-1,3-benzo-thiazol-2-yl)ethyl]-carbamoyl-2-methylpropyl}-carbamate;
(12) Cymoxanil corresponding to 1-(2-cyano-2-methoxyimino-acetyl)-3-ethylurea;
(13) Azoxystrobin corresponding to (E)-2-[2-[6-(2-cyanophenoxy)-pyrimidin-4-yloxy]phenyl-3-methyl methoxyacrylate;
(14) Metominofen corresponding to N-methyl-(E)-methoxyimino-(2-phenoxyphenyl)acetamide;
15) Pyraclostrobin corresponding to methyl N-(2-[1-(4-chlorophenyl)pyrazol-3-yloxymethyl]-phenyl)-N-methoxycarbamate;
(16) Acibenzolar-5-methyl corresponding to methyl benzo (1,2,3) thiadiazole-7-thiocarboxylate;
(17) Famoxadone corresponding to 5-methyl-5-(4-phenoxyphenyl)-3-(phenylamino) oxazolidin-2,4-dione;
(18) Fenamidone corresponding to 4-methyl-4-phenyl-1-(phenylamino)-2-methylthioimidazolidin-5-one; (19) Cyazofamide, corresponding to 2-cyano-4-chloro-5-(4-methylphenyl)-1-(N,N-dimethylamino-sulfamoyl) imidazole; (20) Fluazinam corresponding to 3-chloro-N-(3-chloro-5-trifluoromethyl-2-pyridyl)-α-α-α-trifluoro-2,6-dinitro-p-toluidine;
(21) Dimethomorph corresponding to (E,Z)-4-[3-(4-chlorophenyl)-3-(3,4-dimethoxyphenyl)-acryloyl]morpholine; or Flumorph (SYP-L190) corresponding to (E, Z)-4-[3-(4-fluorophenyl)-3-(3,4-dimethoxyphenyl)-acryloyl]morpholine;
(22) Flumetover corresponding to 2-(3,4-dimethoxyphenyl)-N-ethyl-α,α.α-trifluoro-Nmethylp-toluamide;
(23) Chlorothalonil corresponding to 1,3-dicyano-2,4,5,6-tetrachlorobenzene;
(24) Mancozeb corresponding to the manganese and zinc salt of ethylenejbis (dithiocarbamate) (polymer);
(25) Tolylfluanide corresponding to N-dichloro-fluoromethylthio-N′,N′-dimethyl-N-ptolylsulf-amide;
(26) Folpet corresponding to N-(trichloromethyl-thio) phthalimide;
(27) Etridiazole corresponding to ethyl-3-trichloromethyl-1,2,4-thiadiazolyl ether;
(28) Hymexanol corresponding to 5-methyliso-oxazol-3-ol;
(29) Propatnocarb corresponding to propyl-(3-dimethylaminopropyl) carbamate;
(30) R-3-aminobutanoic acid or RS-3-aminobutanoic acid;
(31) Zoxamide, corresponding to 3,5-dichloro-N-(3-chloro-1-ethyl-1-methyl-2-oxopropyl)-p-toluamide;
32) 2-(4-chlorophenyl)-N-[2-(3-methoxy-4-prop-2-inyloxy-phenyl)ethyl]-2-prop-2-inyloxy-acetamide;
(33) Ethaboxam, (RS)— (α-cyano-2-thienyl)-4-ethyl-2 (ethylamino)-5-thiazolecarboxyamide;
(34) Saccharin.

6. The fungicidal composition according to claim 1, characterized in that the copper (II) salt of phosphorous acid and the other metallic salts of phosphorous acid are present in a ratio ranging from 10/90 to 90/10 in equivalent phosphorous acid.

7. The fungicidal composition according to claim 1, characterized in that the copper (II) salt of phosphorous acid and the other metallic salts of phosphorous acid are present in a 50/50 ratio in equivalent phosphorous acid.

8. The fungicidal composition according to claim 1, characterized in that the concentration of active substances, the copper (II) salt of phosphorous acid, the other metallic salts of phosphorous acid and/or the fungicidal compounds, ranges from 0.1% to 98% by weight, preferably from 0.5% to 90% by weight.

9. The fungicidal composition according to claim 1, characterized in that it consists of a mixture selected from:
Mixture 1: CuHPO₃+CaHPO₃ (50:50)
Mixture 2: CuHPO₃+ZnHPO₃ (50:50)
Mixture 3: CuHPO₃+MnHPO₃ (50:50)
Mixture 4: CuHPO₃+MnHPO₃ (66:33)
Mixture 100: Mixture 1+IR5885 (750⁽¹⁾:15⁽²⁾)•Mixture 103: Mixture 3+IR5885 (750⁽¹⁾:15⁽²⁾)
Mixture 105: Mixture 2+IR5885 (750⁽¹⁾:15⁽²⁾)
Mixture 124: CuHPO₃+IR5885 (750⁽¹⁾:15⁽²⁾)
Mixture 125: CuHPO₃+IR6141 (750⁽¹⁾:15⁽²⁾)
Mixture 126: Mixture 1+IR6141 (750⁽¹⁾:15⁽²⁾)•Mixture 129: Mixture 3+IR6141 (750⁽¹⁾:15⁽²⁾)
Mixture 131: Mixture 2+IR6141 (750⁽¹⁾:15⁽²⁾)
Mixture 143: CuHPO₃+Cu(SA) (750⁽¹⁾:64⁽³⁾)
Mixture 144: Mixture I+Cu(SA) (750⁽¹⁾:64⁽³⁾)
Mixture 145: Mixture 3+Cu(SA) (750⁽¹⁾:64⁽³⁾)•Mixture 146: Mixture 2+Cu(SA) (750⁽¹⁾:64⁽³⁾)
Mixture 152: CuHPO₃+Cu(ASA)₂ (750⁽¹⁾:64⁽³⁾)
Mixture 153: Mixture 1+Cu(ASA)₂ (750⁽¹⁾:64⁽³⁾)
Mixture 154: Mixture 3+Cu(ASA)₂ (750⁽¹⁾:64⁽³⁾)
Mixture 155: Mixture 2+Cu(ASA)₂ (750⁽¹⁾:64⁽³⁾)•Mixture 361: CuHPO₃+Saccharin (750⁽¹⁾:125⁽²⁾)
Mixture 362: Mixture 1+Saccharin (750⁽¹⁾:125⁽²⁾)
Mixture 363: Mixture 2+Saccharin (750⁽¹⁾:125⁽²⁾)
Mixture 364: Mixture 3+Saccharin (750⁽¹⁾:125⁽²⁾)

10. The fungicidal composition according to claim 9, characterized in that it consists of a mixture selected from mixture 1, mixture 2, mixture 3 and mixture 4.

11. A process for the preparation of a composition according to claim 1, characterized in that the mixtures comprising a copper (II) salt of phosphorous acid and at least another metallic salt of phosphorous acid are obtained by mixing the single salts or by co-precipitation of said salts in the reaction mixture.

12. The process according to claim 11, characterized in that the co-precipitation of the salts is effected starting from phosphites, for example sodium, potassium, by the shifting of the metallic cation M₁ with a mixture, in aqueous solution, of a copper (II) salt and the other desired metallic salt, according
to scheme A:
Scheme A
(p+q)M¹₂HPO₃+pCuX+qM²X¹>CuₚM²_{q}(HPO₃)_{(p+q)}+M¹_{2(p+q)}XₚX¹_{q} wherein M¹ represents a sodium or potassium atom, X represents a halogen, such as chlorine, bromine, iodine or a sulfate group, X¹ has the same meaning as X, M² represents an alkaline earth metal, an ammonium group or a Fe, Mn, Zn, Ni, Al, Ti, Se atom; p is a number greater than 0 and lower than 100 and q is equal to 100−p.

13. The process according to claim 11, characterized in that the co-precipitation of a copper (II) salt of phosphorous acid and a salt of an alkaline metal of phosphorous acid is effected starting from the above alkaline salt of phosphorous acid by partial shifting of the metallic cation with a suitable copper (II) salt according to scheme B:
Scheme B
(p+q)M¹₂HPO₃+pCuX>CupM¹2_{q}(HPO₃)_{(p+q)}+M¹₂ₚXₚ wherein M¹, X, p and q have the meanings indicated above./

14. The process according to claim 11, characterized in that the active substance, i.e. the copper (II) salt of phosphorous acid, and other metallic salts of phosphorous acid and/or the fungicidal compounds, is diluted or dissolved in a solvent medium and/or a solid diluent, possibly in the presence of surface-active agents.

15. The use of a fungicidal compound consisting of mixtures comprising a copper (II) salt of phosphorous acid and at least another metallic salt of phosphorous acid or mixtures comprising a copper (II) salt of phosphorous acid, alone or mixed with at least another metallic salt of phosphorous acid, and one or more fungicidal compounds, for the control of phytopathogen fungi.

16. Use according to claim 15, characterized in that the phytopathogens are: Plasmopara viticola (vines); Phytophtora infestans (tomatoes, pota-toes); Phytophtora nicotianae (tobacco, ornamental plants); Phytophtora palmivora (cacao); Phytophtora cinnamomi (pineapples, citrus fruit); Phytophtora capsici (peppers, tomatoes, cucurbits) Phytophtora cryptogea (tomatoes, plums, ornamental plants); Phytophtora megasperma (ornamental plants); Phytophtora citri (citrus fruit); Peronospora tabacina (tabacco); Pseudoperonospora cubensis (cauliflowers, cucurbits); Pseudoperonospora umili (hops); Phytophtora cactorum (fruit trees); Phytophtora destructor (onions); Phytophtora fragarie (strawberries); Bremia (salad).

17. A method for the control of phytopathogen fungi in agricultural crops by the application of a fungicidal composition according to claim 1.

18. The method according to claim 17, characterized in that the fungicidal composition has the following application dosages per hectare: 1,000-4,000 g of salt of phosphorous acid; −5-3,500 g for each fungicidal compound from (1) to (34) present in the composition.

19. The method according to claim 17, characterized in that the application is effected on all parts of the plant, for example on the leaves, stems, branches and roots, or on the seeds themselves before sowing, or on the ground in which the plant grows.

20. The method according to claim 17, characterized in that the application is effected for preventive, protective, prophylactic, systemic, curative and de-rooting treatment.
